SEQUENCE LISTING (80 sequences)

SEQ ID NO: 1 - DNA, 44 bp, Artificial Sequence
  chemically-synthesized DNA sequence
atgcgtcatgtcatgctgatatcagggtatgtctgtcgtacgta

SEQ ID NO: 2 - DNA, 44 bp, Artificial Sequence
  chemically-synthesized DNA sequence
ctgcctatgttcgatgtcctatacattgagtctctttcaggcag

SEQ ID NO: 3 - DNA, 44 bp, Artificial Sequence
  chemically-synthesized DNA sequence
ctgccttctactgctgcaataccatgagtgggcagcttaggcag

SEQ ID NO: 4 - DNA, 44 bp, Artificial Sequence
  chemically-synthesized DNA sequence
ctgcctgtgtagaacgtttttctttcgtagcagttcccaggcag

SEQ ID NO: 5 - DNA, 44 bp, Artificial Sequence
  chemically-synthesized DNA sequence
cccaagaacccgctcgccgtggtgacgtcgatcatgccttttgg

SEQ ID NO: 6 - DNA, 44 bp, Artificial Sequence
  chemically-synthesized DNA sequence
cccaagaacccgctcgccgtggtgacgttgatcatgccttttgg

SEQ ID NO: 7 - DNA, 44 bp, Artificial Sequence
  chemically-synthesized DNA sequence
ctgccttcacattgtgcacacttggttttcatcggtataggcag

SEQ ID NO: 8 - DNA, 44 bp, Artificial Sequence; identical to SEQ ID NO: 6
  chemically-synthesized DNA sequence

SEQ ID NO: 9 - DNA, 44 bp, Artificial Sequence
  chemically-synthesized DNA sequence
ctgcctcacgtatgatatcctctatgacgtatattccgaggcag

SEQ ID NO: 10 - DNA, 44 bp, Artificial Sequence
  chemically-synthesized DNA sequence
cataaacgcagtgacgttcccagaattgtggctgatgattttgg

SEQ ID NO: 11 - DNA, 44 bp, Artificial Sequence; identical to SEQ ID NO: 10
  chemically-synthesized DNA sequence

SEQ ID NO: 12 - DNA, 44 bp, Artificial Sequence
  chemically-synthesized DNA sequence
ctgcctgttactatatctagctcgggtaggcacgtttcaggcag

SEQ ID NO: 13 - DNA, 44 bp, Artificial Sequence; identical to SEQ ID NO: 10
  chemically-synthesized DNA sequence

SEQ ID NO: 14 - DNA, 44 bp, Artificial Sequence
  chemically-synthesized DNA sequence
atattccttaatctgcgcattttacgcatttaactctttgccca

SEQ ID NO: 15 - DNA, 43 bp, Artificial Sequence
  chemically-synthesized DNA sequence
atattccttaatctgcgcattttacgcattaactctttgccca

SEQ ID NO: 16 - DNA, 44 bp, Artificial Sequence
  chemically-synthesized DNA sequence
ctgcctgattctgctctattctcgataaagtttagtagaggcag

SEQ ID NO: 17 - DNA, 44 bp, Artificial Sequence
  chemically-synthesized DNA sequence
ctgcctcagtaaagtcgtcgttaagagggtgacatttcaggcag

SEQ ID NO: 18 - DNA, 44 bp, Artificial Sequence
  chemically-synthesized DNA sequence
ctgcctactgggaggggtcctagcgctgagacgcgtcgaggcag

SEQ ID NO: 19 - DNA, 44 bp, Artificial Sequence
  chemically-synthesized DNA sequence
ctgcctggtctttagtctctattgagtggctaaagttgaggcag

SEQ ID NO: 20 - DNA, 43 bp, Artificial Sequence
  chemically-synthesized DNA sequence
caagagagagagaggggaggagtgggggggggaggatggctgg

SEQ ID NO: 21 - DNA, 44 bp, Artificial Sequence; identical to SEQ ID NO: 6
  chemically-synthesized DNA sequence

SEQ ID NO: 22 - DNA, 46 bp, Artificial Sequence
  chemically-synthesized DNA sequence
ctgcctattaaattttggatttatagcgaccaaagcccgcagcaag

SEQ ID NO: 23 - DNA, 44 bp, Artificial Sequence
  chemically-synthesized DNA sequence
ctgcctcggggggggagggtggcccgggtgggaggtagaggcag

SEQ ID NO: 24 - DNA, 44 bp, Artificial Sequence
  chemically-synthesized DNA sequence
ctgcctcgcggttggacactctggtgggcgggggggggaggcag

SEQ ID NO: 25 - DNA, 44 bp, Artificial Sequence; identical to SEQ ID NO: 10
  chemically-synthesized DNA sequence

SEQ ID NO: 26 - DNA, 44 bp, Artificial Sequence
  chemically-synthesized DNA sequence
ctgcctgtttgagaaattgtataagtgtgaagatcgataggcag

SEQ ID NO: 27 - DNA, 44 bp, Artificial Sequence; identical to SEQ ID NO: 10
  chemically-synthesized DNA sequence

SEQ ID NO: 28 - DNA, 44 bp, Artificial Sequence
  chemically-synthesized DNA sequence
ctgcctgacttcaatatccggcctttgttaatgtgttaaggcag

SEQ ID NO: 29 - DNA, 44 bp, Artificial Sequence
  chemically-synthesized DNA sequence
ctgccttgtcattcactcatcactttgcttttagtagtaggcag

SEQ ID NO: 30 - DNA, 44 bp, Artificial Sequence
  chemically-synthesized DNA sequence
ctgcctttaaggacgaatctttaagatgggtaatcgggaggcag

SEQ ID NO: 31 - DNA, 44 bp, Artificial Sequence
  chemically-synthesized DNA sequence
ctgcctgtgtccatttcctatgagagtggagggactgtaggcag

SEQ ID NO: 32 - DNA, 44 bp, Artificial Sequence; identical to SEQ ID NO: 10
  chemically-synthesized DNA sequence

SEQ ID NO: 33 - DNA, 44 bp, Artificial Sequence
  chemically-synthesized DNA sequence
ctgcctaatcctcttgccagactacatacagtggtcccaggcag

SEQ ID NO: 34 - DNA, 44 bp, Artificial Sequence
  chemically-synthesized DNA sequence
ctgccttggtccttactgaacggggctcagtctccgctaggcag

SEQ ID NO: 35 - DNA, 44 bp, Artificial Sequence
  chemically-synthesized DNA sequence
ctgcctgtatatgttcaacttacgccacagaacctctcaggcag

SEQ ID NO: 36 - DNA, 44 bp, Artificial Sequence
  chemically-synthesized DNA sequence
ctgcctttcgtcttatctcgtctgtggtaaattcaatcaggcag

SEQ ID NO: 37 - DNA, 44 bp, Artificial Sequence
  chemically-synthesized DNA sequence
ctgcctcatgttcgtgtctatccatgcgatatgtcatcaggcag

SEQ ID NO: 38 - DNA, 45 bp, Artificial Sequence
  chemically-synthesized DNA sequence
cccaagaacccgctcgccgtgggtgacgttgatcatgccttttgg

SEQ ID NO: 39 - DNA, 44 bp, Artificial Sequence
  chemically-synthesized DNA sequence
catacacacccaccaaacaattccacgatcttgcacgcttgtcc

SEQ ID NO: 40 - DNA, 44 bp, Artificial Sequence
  chemically-synthesized DNA sequence
ccgaaaatcgaattacacacaccatacatgtacctgcaccttcc

SEQ ID NO: 41 - DNA, 44 bp, Artificial Sequence
  chemically-synthesized DNA sequence
caaacatcacactcagacctcggtaaaataatcccacacactcc

SEQ ID NO: 42 - DNA, 44 bp, Artificial Sequence
  chemically-synthesized DNA sequence
ccgggagagagagagaggagatggcggttggggtgggcggatgg

SEQ ID NO: 43 - DNA, 44 bp, Artificial Sequence
  chemically-synthesized DNA sequence
cccgaacacaccaccgtccttcacatgtttttaccccaccatgc

SEQ ID NO: 44 - DNA, 44 bp, Artificial Sequence
  chemically-synthesized DNA sequence
ggcggaggaagggagaggccgggtggcggggcggacgagcaggg

SEQ ID NO: 45 - DNA, 44 bp, Artificial Sequence
  chemically-synthesized DNA sequence
caggagcagggggagcattttggatgtatggttggtttggttgg

SEQ ID NO: 46 - DNA, 44 bp, Artificial Sequence
  chemically-synthesized DNA sequence
ggcggaggaagggagagggcgggtggcggggcggacgagcaggg

SEQ ID NO: 47 - DNA, 44 bp, Artificial Sequence
  chemically-synthesized DNA sequence
cacacattcacatcacgctctaaatccgattcagtctgctggcc

SEQ ID NO: 48 - DNA, 44 bp, Artificial Sequence
  chemically-synthesized DNA sequence
cgaggggaaggatggggtggagggaggtgggggagggttggtgg

SEQ ID NO: 49 - DNA, 44 bp, Artificial Sequence
  chemically-synthesized DNA sequence
ccacaccgcatgcttactccatactcacattcctttagccctcc

SEQ ID NO: 50 - DNA, 44 bp, Artificial Sequence
  chemically-synthesized DNA sequence
ccaccacactcacggccagaatataatccactccaaaaagttcc

SEQ ID NO: 51 - DNA, 44 bp, Artificial Sequence
  chemically-synthesized DNA sequence
cgtgtgcccttatcaataaatatccttgttacgtgtccctctcc

SEQ ID NO: 52 - DNA, 44 bp, Artificial Sequence
  chemically-synthesized DNA sequence
cgaaaaaaaaaccaagcaagataggggaggggtgggggagggcc

SEQ ID NO: 53 - DNA, 44 bp, Artificial Sequence
  chemically-synthesized DNA sequence
caggaagagaaaaataatgagagagatgaggagtgtggggtgcg

SEQ ID NO: 54 - DNA, 44 bp, Artificial Sequence
  chemically-synthesized DNA sequence
tgacactcgcatacacacatccccccctaagaatcttgatctcc

SEQ ID NO: 55 - DNA, 44 bp, Artificial Sequence
  chemically-synthesized DNA sequence
cattcgagagagagaacggttgtataggtggggcgggtgggtgg

SEQ ID NO: 56 - DNA, 44 bp, Artificial Sequence
  chemically-synthesized DNA sequence
cccggacacacacaccttctcgtaatttgcatatcactcctccc

SEQ ID NO: 57 - DNA, 44 bp, Artificial Sequence
  chemically-synthesized DNA sequence
ccgggagagagggagaggagatggcggttggggtgggcggatgg

SEQ ID NO: 58 - DNA, 43 bp, Artificial Sequence
  chemically-synthesized DNA sequence
gagcggaggtgggtgagggaggaggggaggggatttgctgggc

SEQ ID NO: 59 - DNA, 44 bp, Artificial Sequence
  chemically-synthesized DNA sequence
cagtggagggtgtggagggatgtgggtgggtggtgtgggtgtgg

SEQ ID NO: 60 - DNA, 44 bp, Artificial Sequence
  chemically-synthesized DNA sequence
ccccatccccttaaaagtgcacatatcaaacacccctccatccc

SEQ ID NO: 61 - DNA, 44 bp, Artificial Sequence
  chemically-synthesized DNA sequence
cccacaccttggcccgaaaacagaccacacacccgacctcatcc

SEQ ID NO: 62 - DNA, 44 bp, Artificial Sequence; identical to SEQ ID NO: 46
  chemically-synthesized DNA sequence

SEQ ID NO: 63 - DNA, 44 bp, Artificial Sequence; identical to SEQ ID NO: 6
  chemically-synthesized DNA sequence

SEQ ID NO: 64 - DNA, 44 bp, Artificial Sequence; identical to SEQ ID NO: 6
  chemically-synthesized DNA sequence

SEQ ID NO: 65 - DNA, 44 bp, Artificial Sequence
  chemically-synthesized DNA sequence
cacctccttgtcatatggcttctctctctctgggtttgctgtgc

SEQ ID NO: 66 - DNA, 44 bp, Artificial Sequence
  chemically-synthesized DNA sequence
ccagactttgtgccccatcctaccacccatcatcccctgcttcc

SEQ ID NO: 67 - DNA, 44 bp, Artificial Sequence; identical to SEQ ID NO: 46
  chemically-synthesized DNA sequence

SEQ ID NO: 68 - DNA, 43 bp, Artificial Sequence
  chemically-synthesized DNA sequence
ggggaggaggtgtggcggtgggggaggggatggtggtcgttgg

SEQ ID NO: 69 - DNA, 43 bp, Artificial Sequence
  chemically-synthesized DNA sequence
ccccatccccttaaagtgcacatatcaaacacccctccatccc

SEQ ID NO: 70 - DNA, 44 bp, Artificial Sequence; identical to SEQ ID NO: 46
  chemically-synthesized DNA sequence

SEQ ID NO: 71 - DNA, 44 bp, Artificial Sequence
  chemically-synthesized DNA sequence
cacatccatagccgttagttccgtttatcaccacatctccttcc

SEQ ID NO: 72 - DNA, 44 bp, Artificial Sequence; identical to SEQ ID NO: 71
  chemically-synthesized DNA sequence

SEQ ID NO: 73 - DNA, 42 bp, Artificial Sequence
  chemically-synthesized DNA sequence
ccgggagagagagaggagatggcggttggggtgggcggatgg

SEQ ID NO: 74 - DNA, 44 bp, Artificial Sequence
  chemically-synthesized DNA sequence
ccccaaatacacctttcgacacattctcggctcgtcatgtcacc

SEQ ID NO: 75 - DNA, 42 bp, Artificial Sequence
  chemically-synthesized DNA sequence
accaaacacaaacagagagagagagggggagggagggggggc

SEQ ID NO: 76 - DNA, 45 bp, Artificial Sequence
  chemically-synthesized DNA sequence
gggggaggttgggcgaggggacggtcgggtggtgtcggtgttggg

SEQ ID NO: 77 - DNA, 44 bp, Artificial Sequence
  chemically-synthesized DNA sequence
cgcacacacactcatgcattcagaccatcgtatccctttcatcc

SEQ ID NO: 78 - DNA, 45 bp, Artificial Sequence
  chemically-synthesized DNA sequence
ggcggaggaagggagagggcgggtggcggggccggacgagcaggg

SEQ ID NO: 79 - DNA, 44 bp, Artificial Sequence
  chemically-synthesized DNA sequence
catttggtgagtgtggatagagaggggagggtgtgggtgggtcc

SEQ ID NO: 80 - DNA, 44 bp, Artificial Sequence
  chemically-synthesized DNA sequence
caaaacaccacacacttcacaccccaataatcaattctgtcacc

(19) United States
(12) Patent Application Publication
(10) Pub. No.: US 2020/0283774 A1
(43) Pub. Date: Sep. 10, 2020

(54) APTAMER AGAINST M.TB MPT51 AND USES THEREOF

(71) Applicants: TRANSLATIONAL HEALTH SCIENCE AND TECHNOLOGY INSTITUTE, Faridabad (IN); ALL INDIA INSTITUTE OF MEDICAL SCIENCES, New Delhi (IN); APTABHARAT INNOVATION PRIVATE LIMITED, Northeast, Delhi (IN)

(72) Inventors: Tarun KUMAR SHARMA, Faridabad (IN); Jaya SIVASWAMI TYAGI, New Delhi (IN); Ritu DAS, Delhi (IN); Abhijeet DHIMAN, New Delhi (IN)

(73) Assignees: TRANSLATIONAL HEALTH SCIENCE AND TECHNOLOGY INSTITUTE, Faridabad (IN); ALL INDIA INSTITUTE OF MEDICAL SCIENCES, New Delhi (IN); APTABHARAT INNOVATION PRIVATE LIMITED, Northeast, Delhi (IN)

(21) Appl. No.: 16/758,185

(22) PCT Filed: Sep. 7, 2018
(86) PCT No.: PCT/IN2018/050581

§ 371 Date: Apr. 22, 2020

(30) Foreign Application Priority Data
Jun. 22, 2018 (IN) 201811023360

(51) Int. Cl. C12N 15/115; C12Q 1/68; C12Q 1/04

(52) U.S. Cl. CPC C12N 15/115; C12N 2310/16; C12Q 1/04; C12Q 1/68

(57) ABSTRACT

The invention provides single stranded DNA aptamers specific to M.tb MPT51 and uses thereof in rapid, robust, highly specific, and cost effective diagnosis of tuberculosis. The invention also provides methods and devices based on the aptamers of the invention for the diagnosis of tuberculosis. Advantageously, aptamers of the present invention can selectively detect as low as 2 ng of M.tb MPT51 and this activity remains unaltered in presence of anti-MPT51 antibodies. The developed device can give sample-to-answer within 30 minutes.

### TECHNICAL FIELD

The present invention relates to aptamers specific to M.tb biomarker MPT51 and uses thereof in diagnosis of tuberculosis in human.

### BACKGROUND ART

Mycobacterium tuberculosis is the causative agent of one of the world's most malicious diseases, affecting almost one-third population of the world (˜2.7 billion) suffers from TB (WHO, 2017). In 2016, more than 10 million people suffered from active TB, for this reason, TB still holds a number one position as a deadliest infectious disease. The emergence of drug-resistant strains of TB and co-infections with human immunodeficiency virus (HIV) might also add-on this burden globally. Mycobacterium tuberculosis (M.tb) is a slow growing, obligate aerobe, non-motile, non-spore forming, and non-capsulate straight or slightly curved rod shaped bacterium. On the basis of site of infection TB is divided into two categories namely; Pulmonary (in about 90% cases it involves Lungs, and nearly 25% of people do not reflect any symptoms i.e. they remain “asymptomatic) and Extra pulmonary (when infection outburst other than lungs which involves Pleura, Central Nervous System (CNS), Lymphatic systems, bones and joints, called as extrapulmonary tuberculosis). Tuberculous meningitis (IBM) is the most dreaded manifestation of tuberculosis that causes irreversible neurological damage to CNS. However, it is treatable with the current drug regimen but require early and accurate detection.

Moreover due to the emergence of MDR-TB (multi drug resistant TB) and XDR-TB (extensively drug resistant TB) which are the two forms of TB in which bacteria do not respond first line and second line anti TB drugs respectively, it is important to diagnose the TB accurately and timely at an early stage.

There is an urgent need to device new diagnostic reagents, methods and tools to device rapid, specific and robust but low-cost detection of tuberculosis.

Aptamers have emerged as a potential rival for antibodies in therapeutics, diagnostics and bio-sensing due to their inherent characteristics. Aptamers specific to various M.tb antigens have been designed worldwide for diagnosis of tuberculosis. India is also not untouched in this regard. Indian Patent application No. 201611001550 provides for single stranded DNA aptamers specific to M.tb HspX antigen and uses thereof in diagnosis of tuberculosis. Another Indian Patent Application no.201611021901 provides for single stranded DNA aptamers specific to M.tb GlcB antigen and uses thereof in diagnosis of tuberculosis.

Accurate diagnosis of TB requires reliable biomarkers as target of detection. One such target is 27KDa protein or MPT51 (RV3803c) which is known to exhibit more than 60% sequence similarity in its N-terminal region with the antigen 85 complex proteins. The MPT51 protein belongs to the family of a/B non-catalytic hydrolases that may also be involved in bacterial adhesion to the extracellular matrix. MPT51 is known to express in early stage of M.tb infection independent of HIV-Co infection. Its utility was demonstrated in the detection of TB meningitis using CSF samples. MPT51 evinced >90% sensitivity and specificity. Laal et al., 2006 explored immune diagnosis of pulmonary TB using this M.tb antigen in HIV negative and HIV positive TB cases.

Panels, methods, devices, reagents, systems, and kits utilizing anti-MPT51 antibodies have been designed worldwide. For example, PCT Application No. 20020024297 provides for immunoassays detecting MPT51 antibodies in subject's urine. US patent application no. 20180136207 provides immunoassay in blood, serum, plasma, urine, pleural fluid, ocular fluid or saliva of the subject detecting MPT51 antibodies. US patent application no.20090280140 provides for an early detection method for tuberculosis using peptides from immune-dominant antigens GlcB and MPT51. PCT application No. 2014059336 provides for diagnosis of tuberculosis using anti-MPT51 antibodies.

There are few patent applications on aptamers for detection of MPT51. For example, PCT application No. 2007005627 provides for a method for diagnosis of tuberculosis by detecting presence of hybridization between oligonucleotide probes and M.tb antigens present in the biological sample. Another PCT application no. 2015164617 provides a method of detecting tuberculosis infection in a subject, comprising detecting at least one M.tb biomarker selected from MP64, ACR, CH602, PstS1, DnaK, MASZ, CHIO, RL7, TPX, CF30, KAD, MPT51, EsxB, EsxA, A85 A, A85B, and A95C in a urine sample from the subject using slow off-rate aptamers having upto 10 nucleotides as biomarker capture reagent but there is no information disclosure regarding limit of detection and OD values.

In present invention, applicants have engineered highly specific aptamers for M.tb MPT51 which can be used for designing methods and devices for fast, reliable, and affordable diagnosis of tuberculosis in human.

### SUMMARY OF THE INVENTION

The present invention is directed to providing an aptamer for M.tb MPT51. The present invention is also directed to method and device utilizing the M.tb MPT51 aptamers of the invention for the diagnosis of tuberculosis in humans.

The present inventors investigated diligently to solve the problem described above and succeeded in preparing an aptamer of good quality for M.tb MPT51, which resulted in the completion of the present invention.

Accordingly, the present invention provides the following:
(i) A single stranded DNA aptamer that binds to M.tb MPT51 and that comprises the single stranded DNA sequence or a complementary DNA sequence thereof, or a truncated portion thereof, or any pairing thereof, or any modification thereof, is homologous of any of: (a) SEQ ID NO: 1 to SEQ ID NO:80 (also mentioned as “MPT51 aptamers”); or (b) a functional fragment of any preceding sequences;
(ii) A complex comprising an aptamer of (i) and a functional substance;
(iii) A complex of (ii), wherein the, or functional substance is an affinity substance or a substance for labeling;
(iv) A diagnostic reagent comprising an aptamer of any of (i), (ii) or (iii);
(v) A M.tb MPT51 detection probe comprising an aptamer of any of (i), (ii) or (iii);
(vi) An electrochemical device for the detection of M.tb MPT51 in a biological sample obtained from a subject suspected of M.tb infection using the aptamer of (i), (ii) or (iii);
(vii) A method for detection of M.tb MPT51 in a biological sample obtained from a subject suspected of M.tb infection using (i), (ii), (iii) or (vi), wherein tuberculosis in the subject is confirmed by the presence of M.tb MPT51 in the said biological sample.

The aptamer or the complex of the present invention can be useful as diagnostic reagent in diagnosis of tuberculosis. The aptamer or the complex of the present invention can also be useful in purifying and concentrating M.tb MPT51, labeling M.tb MPT51, and detecting and quantifying M.tb MPT51.

Nucleotide sequence of the MPT51 aptamers is referred to herein by a sequence identifier number (SEQ ID NO:) and provided in separate sheet. The SEQ ID NOs: correspond numerically to the sequence identifiers in the sequence listing, eg. SEQ ID NO:1, SEQ ID NO: 2, etc.

### BRIEF DESCRIPTION OF THE DRAWINGS

In the drawings:

FIG. 1 provides general schema of SELEX Incubation of random ssDNA library with Nitrocellulose membrane (negative selection) (step a); removal of bound DNA and retain unbound DNA (step b); positive selection with MPT51 immobilized on Nitrocellulose membrane and bound DNA was retained after washing (step c); and binders were enriched using PCR (step d). The enriched DNA obtained after 10 rounds of this iterative process was cloned and sequenced to identify the sequences of monoclonal aptamers (step e).

FIG. 2(A) Agarose gel (2%), electrophoretogram showing amplification of aptamers atdifferent number of cycles, lane 1 (5 cycles), lane 2 (8 cycles), lane 3 (10 cycles), lane 4 (12 cycles), lane 5 (15 cycles), lane 6 (20 cycles), lane 7 (25 cycles) and lane 8 (−ve control). (B) Electrophoretogram of 10% Urea-PAGE showing 80 nucleotide (nt) FAM labeled ssDNA.

FIG. 3 provides general schema of Aptamer Linked Immunosorbent Assay (ALISA). (A) Coating of MPT51(500 ng/well) on 96 well plate, (B) Addition of biotinylated aptamer candidate (100 pmole/well), (C) After washing away of unbound aptamer, streptavidin-horse radish peroxidase (HRP) was added, (D) 3, 3′, 5, 5′-tetramethylbenzidine (TMB) substrate was added to monitor the aptamer binding to MPT51, (E) Oxidation of TMB was followed as a color change from colorless to blue.

FIG. 4 provides representation of the binding efficiency of aptamers from different rounds of population. RDL(Random DNA library); R2, R4, R6, R7, R8, R9 and R10. R followed by number represents the SELEX round.

FIG. 5 provides representation of relative binding of different monoclonal MPT51 aptamers. MPT51 aptamers colored in red were selected for further characterization based on a cut off of O.D.≥0.5.

FIG. 6 provides representation of detection of MPT51 by the selected MPT51 aptamers using dot-blot assay.

FIG. 7 provides representation of Heat Map matrix showing the z-score of the absorbance response for individual GMPT51 aptamer/anti-MPT51 antibody (Ab) against tested antigens.

FIG. 8 provides representation of the outcome of aptamer-antibody competition assay. (A) ALISA was performed in aptamer favorable condition to assess the binding of MPT51 aptamers to MPT51 in the presence of poly and monoclonal anti-MPT51 antibody. (B) ALISA was performed in antibody favorable condition to assess the binding of MPT51 aptamers in the presence of poly and monoclonal anti-MPT51 antibody. Polyclonal antibody was generated in-house while monoclonal was procured form BEI resources-USA

FIG. 9 Evince the limit of detection of MPT51 aptamers. Red arrows denote the limit of detection determined.

FIG. 10 provides representation of apparent dissociation curve (K_{d}) of MPT51 aptamers.

FIG. 11 provides (A) Schematic representation of structural-switching POC electrochemical aptasensor for TBM detection (B) Cyclic voltammograms in 5 mMFe(CN)₆^{3-/4-} containing 0.1 M KCl at 10 mV/s of different modified SPE.

FIG. 12 provides (A)Sensor response to MPT51: A plot of difference in peak current from Differential Pulse Voltammetry (DPV) before and after incubation with different concentration of target protein clearly showing a highly sensitive detection of MPT51 (B) Sensor response to MPT51 in CSF background: A plot of difference in peak current from DPV before and after incubation with different concentration of target protein clearly showing a highly sensitive detection of MPT51 in CSF background.

FIG. 13 provides representation of three color gradient heat map representing the specificity of the sensor

FIG. 14 provides (A) a scatter plot showing highly discriminatory response of sensor for TBM and Not-TBM CSF samples. (B) A scatter plot showing sensor response for various categories of IBM samples (Definite, probable and possible) and Non-TBM samples (NTIM, IND and NIND).

### MODES FOR EMBODYING THE INVENTION

The present invention relates to aptamers specific to M.tb MPT51 and uses thereof in diagnosis of tuberculosis in human. In various embodiments, the invention relates to reagents, methods, and kits or detection of M.tb MPT51 in the biological sample from an individual suspected of tuberculosis.

Accordingly, the present invention provides the following:
(i) A single stranded DNA aptamer that binds to M.tb MPT51 and that comprises the single stranded DNA sequence or a complementary DNA sequence thereof, or a truncated portion thereof, or any pairing thereof, or any modification thereof, is homologous of any of: (a) SEQ ID NO: 1 to SEQ ID NO:80 (also mentioned as “MPT51 aptamers”); or (b) a functional fragment of any preceding sequences;
(ii) A complex comprising an aptamer of (i) and a functional substance;
(iii) A complex of (ii), wherein the, or functional substance is an affinity substance or a substance for labeling;
(iv) A diagnostic reagent comprising an aptamer of any of (i), (ii) or (iii);
(v) A M.tb MPT51 detection probe comprising an aptamer of any of (i), (ii) or (iii);
(vi) An electrochemical device for the detection of M.tb MPT51 in a biological sample obtained from a subject suspected of M.tb infection using the aptamer of (i), (ii) or (iii);
(vii) A method for detection of M.tb MPT51 in a biological sample obtained from a subject suspected of M.tb infection using (i), (ii), (iii) or (vi), wherein tuberculosis in the subject is confirmed by the presence of M.tb MPT51 in the said biological sample.

Assays directed to the detection and quantification of physiologically significant molecules in biological samples and other samples are important tools in scientific research and in the health care field.

An aptamer can be identified using any known method, including the SELEX process. Once identified, an aptamer can be prepared or synthesized in accordance with any known method, including chemical synthetic methods and enzymatic synthetic methods.

The term MPT51 or mycobacterial MPT51 or MPT51 protein or MPT51 or FbpC1 or MPB51 used in this description refers to a protein refer to Mycobacterium tuberculosis gene Rv3803c, which encodes the secreted antigen protein MPT51 (also referred to as the fibronectin-binding protein) which is 27KDa non-catalytic alpha/beta hydrolase having role in host tissue attachment, whereby ligands may include the serum protein fibronectin and small sugars. In addition to being produced in M.tb, M.tb MPT51 as used in the present invention can be prepared using mouse and other mammalian cells, insect cells, cells of Escherichia coli and the like, and can also be prepared by chemical synthesis. When M.tb MPT51 is prepared by cell culture or chemical synthesis, a mutant can easily be prepared. Here, a mutant means a sequence wherein several amino acids have been substituted or a partial amino acid sequence, and means a protein or peptide having at least one of the activities essentially possessed by M.tb MPT51. When an amino acid is substituted, the substituent amino acid may be a naturally occurring amino acid or may be a non-naturally occurring amino acid. As mentioned in the present invention, M.tb MPT51 includes these mutants.

The present invention also provides a solid phase carrier having the aptamer or the complex of the present invention immobilized thereon. As examples of the solid phase carrier, a substrate, a resin, a plate (e.g., multiwell plate), a filter, a cartridge, a column, and a porous material can be mentioned. The substrate can be one used in DNA chips, protein chips and the like; for example, nickel-PTFE (polytetrafluoroethylene) substrates, glass substrates, apatite substrates, silicon substrates, alumina substrates and the like, and substrates prepared by coating these substrates with a polymer and the like can be mentioned. As examples of the resin, agarose particles, silica particles, a copolymer of acrylamide and N,N′-methylenebisacrylamide, polystyrene-crosslinked divinylbenzene particles, particles of dextran crosslinked with epichlorohydrin, cellulose fiber, crosslinked polymers of aryldextran and N,N′-methylenebisacrylamide, monodispersed synthetic polymers, monodispersed hydrophilic polymers, Sepharose, Toyopearl and the like can be mentioned, and also resins prepared by binding various functional groups to these resins were included. The solid phase carrier of the present invention can be useful in, for example, detecting and quantifying M.tb MPT51.

The aptamer or the complex of the present invention can be immobilized onto a solid phase carrier by a method known per se. For example, a method that introduces an affinity substance (e.g., those described above) or a predetermined functional group into the aptamer or the complex of the present invention, and then immobilizing the aptamer or complex onto a solid phase carrier via the affinity substance or predetermined functional group can be mentioned. The present invention also provides such methods. The predetermined functional group can be a functional group that can be subjected to a coupling reaction; for example, an amino group, a thiol group, a hydroxyl group, and a carboxyl group can be mentioned. The present invention also provides an aptamer having such a functional group introduced thereto.

The aptamer or complex of the present invention can be utilized as a detection probe, particularly as a probe for detection of M.tb MPT51. The method of labeling the aptamer is not particularly limited; methods known per se can be applied. Such methods include, for example, labeling with a radioisotope, labeling with a fluorescent dye or fluorescent protein, and the like.

The present invention also provides a method of detecting and quantifying M.tb MPT51. In particular, the present invention makes it possible to detect and quantify M.tb MPT51 separately from other M.tb proteins. The method of detection and quantitation of the present invention can comprise measuring M.tb MPT51 by utilizing the aptamer of the present invention (e.g., by the use of the complex and solid phase carrier of the present invention). The method of detecting and quantifying M.tb MPT51 can be performed in the same manner as an immunological method, except that the aptamer of the present invention is used in place of an antibody. Therefore, by using the aptamer of the present invention as a probe in place of an antibody, in the same manner as such methods as enzyme immunoassay (EIA) (e.g., direct competitive ELISA, indirect competitive ELISA, sandwich ELISA), radioimmunoassay (RIA), fluorescent immunoassay (FIA), use in place of a secondary antibody in Western blot technique, immune-histochemical staining method, and cell sorting method, detection and quantitation can be performed. These methods can be useful in detecting M.tb MPT51 contents in biological samples for diagnosing TB.

### EXAMPLES

### Example 1 SELEX Based Screening of MPT51 Aptamers

### Step 1SELEX

Synthetic ssDNA random library (80-mer) randomized at 44 nucleotides (5′GTC TTGACTAGTTACGCC-N44-GAGGCGCCAACTGAATGA 3′) was custom synthesized by Integrated DNA Technologies (USA). The random region of library was flanked by primer binding sequences to enable DNA amplification by PCR using DRF (Forward-5′ GTC TTG ACT AGT TAC GCC 3′ and DRR (Reverse-5′ TCA AGT TGG CGC CTCA 3′) primers. To prepare ssDNA, PCR was performed using 5′ FAM-labeled DRF and 3′ rA-modified DRR followed by NaOH treatment. Strand separation was achieved on 10% denaturing Urea-PAGE. The SELEX strategy was utilized to engineer MPT51 aptamers.

Briefly; the synthetic ssDNA library (2000 pmol) was heated at 92° C. followed by snap chilling on ice and bringing it to room temperature (RT). The prepared library was incubated with Nitrocellulose membrane (NCM) alone thus, eliminating the non-specific binders (negative SELEX) The unbound pool of aptamers was then incubated with pre-immobilized His6-tagged Purified recombinant MPT51 from M.tb strain H37Rv (2 μg) on NCM in selection buffer (10 mMTris pH 7.5 supplemented with 10 mM MgCl₂, 50 mMKCl, 25 mMNaCl, 0.05% Tween-20 (v/v), 0.1 μg/μL yeast tRNA and 0.1 μg/μL BSA). The unbound ssDNA was washed away with selection buffer (SB) supplemented with 0.5% Tween-20. The bound DNA was eluted by adding nuclease free water (200 μl) and heated at 92° C. for 10 min. The eluted DNA pool was used as template in a PCR reaction containing along with PCR master mix containing forward and reverse primers for an appropriate number of thermal cycling; 94° C. for 30 sec, 55° C. for 30 sec and 72° C. for 30 sec with a final extension at 72° C. for 5 min. The obtained PCR product was precipitated with ethanol, treated with 90 μL of 0.25 N NaOH and heated at 92° C. for 10 min. The alkali-treated DNA pool was then neutralized using sodium acetate (3 M pH 5.5) and precipitated with ethanol. Further, ssDNA was obtained by running this prepared DNA pool on 10% denaturing Urea-PAGE. ssDNA was then eluted from the gel slice in 500 μL elution buffer (10 mMTris pH 7.4 supplemented with 200 mMNaCl and 1 mM EDTA) and kept at 37° C. overnight, precipitated and used in the next round of selection. The stringency of selection was increased in every successive round of SELEX. In order to minimize cross-reactivity, counter selection was also employed by incubating the binder population with other Mtb proteins.

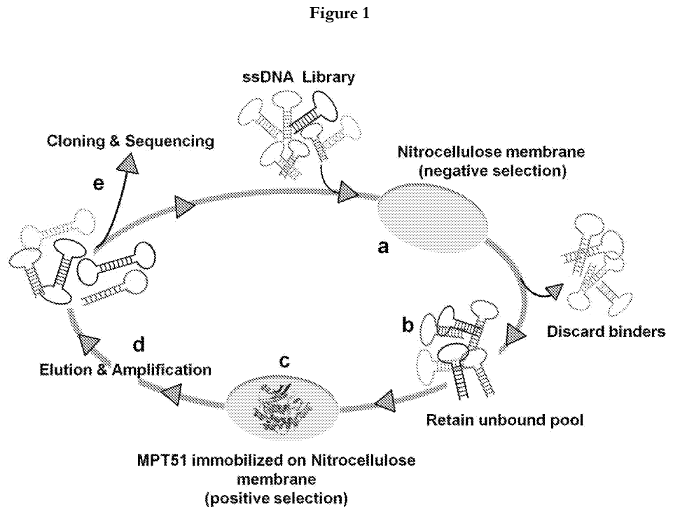

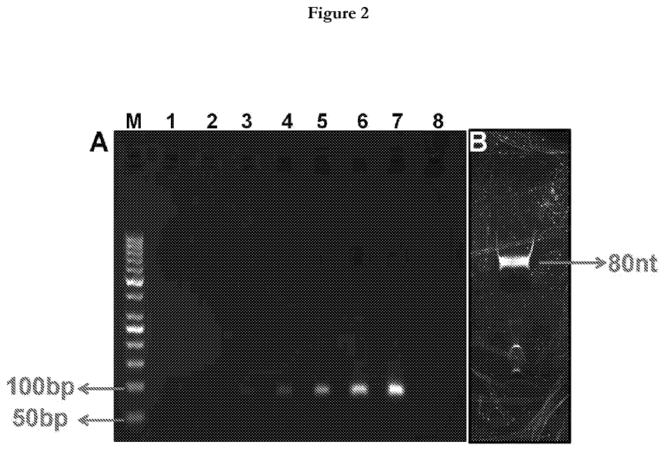

After 10th rounds of selection, the highest affinity pools (round 2nd, 8th and 10th) was cloned in pTZ57R/T vector system (ThermoScientific™InsTAClone PCR cloning Kit) and transformed in E. coli DH5α. The obtained colonies were randomly picked and analyzed by DNA sequencing. The SELEX process is depicted in FIG. 1. A representative electrophoretogram of polymerase chain reaction (PCR) amplification and FAM labeled ssDNA purification is depicted in FIG. 2.

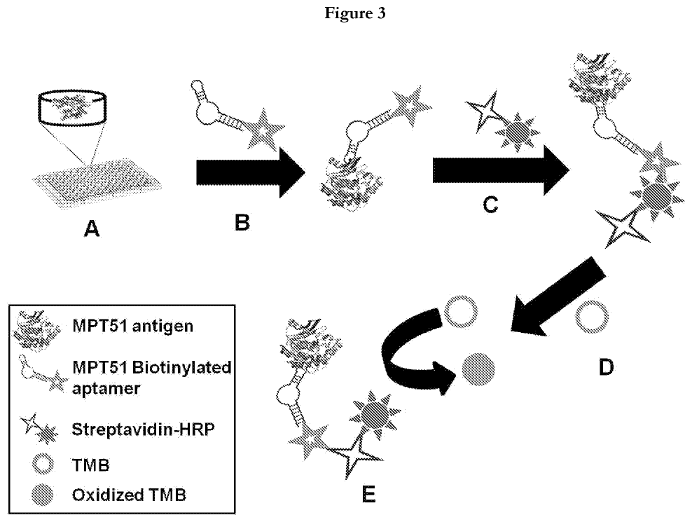

After ten iterative rounds of SELEX, the archived aptamer populations from rounds 2, 4, 6, 7, 8, 9 and 10 were amplified through PCR using biotinylated forward primer and rA containing reverse primer followed by strand separation using denaturing Urea-PAGE to obtain purified ssDNA aptamers. These ssDNA pools of different rounds were checked for their binding to M.tb MPT51 by using Aptamer-Linked Immuno Sorbent Assay (ALISA) method depicted in FIG. 3, where 500 ng of M.tb MPT51 was coated on a 96-well plate overnight at 4° C. One hundred ng of biotinylated binders of the archived population of each round was added to antigen-coated individual wells. Streptavidin-HRP (1:1000 dilution) was then added and the color was developed using TMB (3, 3′, 5, 5′ Tetramethylbenzidine). The reaction was stopped by 5% sulfuric acid (H₂SO₄) followed by absorbance measurement at 450 nm. The O.D. was plotted vs. round of SELEX to assess the enrichment of MPT51 aptamers over successive rounds.

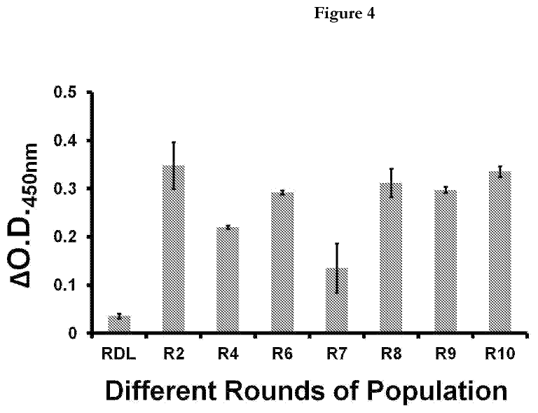

A total 10 rounds of SELEX was performed, then the archived population from different rounds (2, 4, 6, 7, 8, 9 and 10) along with Random DNA Library (RDL) was evaluated for their ability to bind M.tb MPT51 in an Aptamer Linked Immunosorbent Assay (ALISA) based assay. For ALISA, the archived population was first biotinylated using 5′-Biotin labeled primer. FIG. 4 depicts the graph where ΔO.D. at 450 nm is plotted as a function of binding of aptamer pool from different rounds of SELEX Round 2, 8 and 10 exhibited highest O.D. value, implying that the best binders are present in these three rounds therefore; binders from these rounds (rounds 2, 8 and 10) were cloned and sequenced. However, binding of round 8^{th} and 10^{th} populafion is lower than that of round 2^{nd}. This behavior may be due to a decrease in the aptamer pool complexity, inefficient partitioning of bound from unbound sequences during the 8th and 10th round of selection process, or a combination of both. This observation is in concordance with previously published reports. [Hamula et al., 2011].

For cloning, archived binders of Rounds 2, 8 and 10 were amplified by PCR and cloned in pTZ57R/T vector system using InsTACloneTA cloning kit (Thermo scientific) as per manufacturer's instructions. Because the SELEX process utilized a DNA library that was randomized in the central 44 nucleotide region, while the flanking primer binding regions are common in all molecules, therefore, after sequencing the sequence diversity of the random region was examined. In total ˜250 clones were obtained after transformation and out of those, 150 clones were randomly picked and patched on LB agar. One hundred clones were sent for sequencing out of which 80 sequences were passed and rest of the reaction was failed during sequencing. Thus, we have determined the primary sequence homology of as obtained 80 aptamers using ClustalW and Bioedit analysis. Based on the homology results 13 MPT51 aptamers were synthesized by IDT USA.

### Example 2: Screening of Selected Aptamers for Binding (Determination of Absorbance (A450) Value) Through Aptamer Linked Immunosorbent Assay (ALISA)

13 MPT51 aptamers were evaluated for their ability to bind with M.tb MPT51 in the ALISA format. Based on primary sequence nucleotide richness, these MPT 51 aptamers were categorized in two categories: Non-G-Rich and G-Rich. There were 6 Non-G-Rich MPT 51 aptamers and 7 G-Rich MPT51 aptamers.

For screening using ALISA, 500 ng of M.tb MPT51 was coated on 96-well plate (Maxisorp, Nunc) in coating buffer (carbonate-bicarbonate buffer pH 9.6) whereas, a well containing only coating buffer was served as an antigen control. The plate was kept overnight at 4° C. and then was blocked with 5% BSA supplemented with 0.25% Tween-20, for 90 minutes at room temperature (RT). Subsequently, the plate was washed with selection buffer with subsequent addition of 100 picomoles per well was added and incubated for 1 hour at RT. The plate was washed three times with selection buffer containing 1% Tween-20 with subsequent addition of Streptavidin-HRP (1:4000) and incubated for 1 hour. After incubation, washing was done in similar fashion as described in the previous step. MPT51-bound aptamer complex was determined by TMB (3, 3′, 5, 5′ Tetramethylbenzidine) and after color development; the reaction was stopped by 5% sulfuric acid followed by absorbance measurement at 450 nm. The protein-aptamer-strep-complex was quantified at 450 nm (O.D.450) using M2e plate reader (Molecular Devices USA). In this experiment, buffer-coated well served as an antigen control.

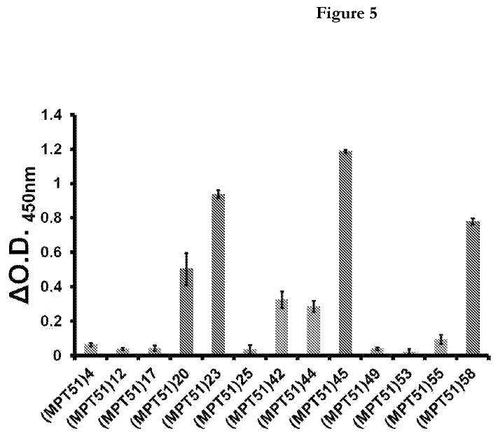

After screening of 13 MPT51 aptamers through ALISA, 4 MPT51 aptamers namely SEQ ID No: 20 or (MPT51)20, SEQ ID No:23 or (MPT51)23, SEQ ID No:45 or (MPT51)45, and SEQ ID No:58 or (MPT51)58 were shortlisted based on a cut-off i.e. ΔO.D.>0.5) for further study as depicted in FIG. 5.

### Example 3: Aptamer-Based Dot Blot Assay for the Detection of M.Tb MPT51

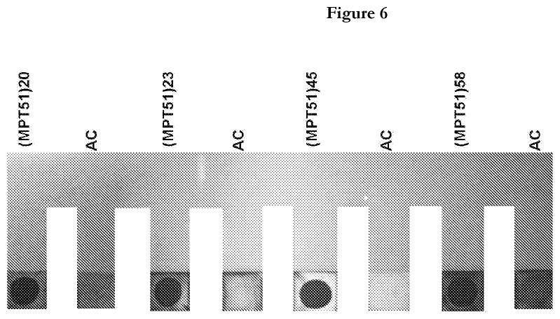

Towards standardization, a visual assay (qualitative) in the form of dot-blot assay has been developed for the detection of M.tb MPT51 by MPT51 aptamers namely, SEQ ID No: 20 or (MPT51)20, SEQ ID No:23 or (MPT51)23, SEQ ID No:45 or (MPT51)45, and SEQ ID No:58 or (MPT51)58. For this, 500 ng of M.tb MPT51 was fixed onto nitrocellulose membrane comb (MDI, Ambala, India) and where no antigen was fixed served as an antigen control (AC) and allowed to dry at RT. The membrane was blocked with 5% BSA for 90 minutes at RT and after incubation the comb was washed twice with SB and allowed to dry at RT. Subsequently, 100 pmol of Biotinylated MPT51 aptamers was added to each arm of the comb and kept for another 1 hour at RT. Thereafter, the comb was washed three times with SB supplemented with 1% Tween-20 followed by the addition of Streptavidin-HRP (1:4000) and incubated for 30 minutes at RT. The comb was washed as above and the blot was developed using metal enhancer DAB (Sigma Aldrich). As a result of dot-blot assay, a visual bluish-black dot emerged within seconds where M.tb MPT51 was fixed and no color was observed on AC arm of the comb as depicted in FIG. 6 which exhibited the quality of the dot-blot assay. Finally, the strip was rinsed with tap water and an image of the dried strip was captured by a digital camera.

### Example 4: Aptamer-Antibody Specificity (Cross-Reactivity) Assay

The MPT51 aptamers namely SEQ ID No: 20 or (MPT51)20, SEQ ID No:23 or (MPT51)23, SEQ ID No:45 or (MPT51)45, and SEQ ID No:58 or (MPT51)58 were tested in ALISA format against 7 different mycobacterial antigens namely HspX, CFP-10, ESAT-6, Ag85C, GroES, Culture filtrate proteins (CFPs), and LAM apart from M.tb MPT51. To examine the cross reactivity of these MPT51 aptamers, 500 ng of each antigenic M.tb protein was coated on to a 96 well plate in 100 μL of coating buffer and kept at 4° C. overnight and rest of the ALISA procedure was performed in the same manner as described in Example 2. A heat map was generated as a function of absorbance of each MPT51 aptamer/anti-MPT51 antibody for tested M.tb antigenic proteins. The color scale of heat map is measured in z-score. The highest z-score (1.5) represents the highest absorbance (ΔO.D.450) while lowest z-score represent the lowest absorbance (ΔO.D.450) value (0.1). Red color represents the highest binding, yellow evince moderate while blue represents the lowest binding.

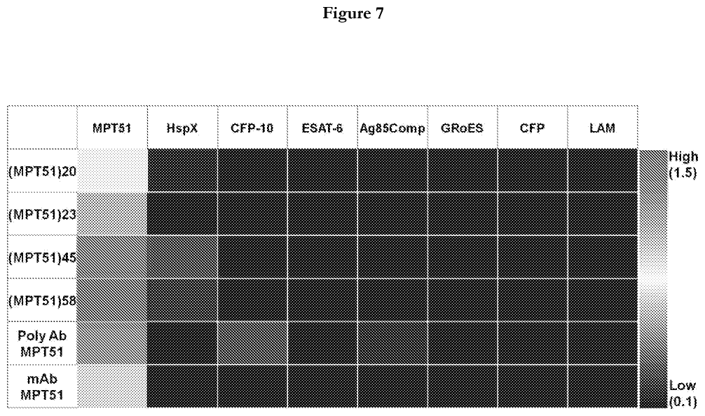

All the four MPT51 aptamers, namely SEQ ID No: 20 or (MPT51)20, SEQ ID No:23 or (MPT51)23, SEQ ID No:45 or (MPT51)45, and SEQ ID No:58 or (MPT51)58 exhibited efficient binding to M.tb MPT51 with minimal or no cross-reactivity with the other antigens tested in the ALISA assay. The binding of individual MPT51 aptamer to the tested antigens is shown in FIG. 7(red colored box depicts the highest binding while blue represent the lowest binding). A comparison of polyclonal and monoclonal anti-MPT51 antibody with these four MPT51 aptamers indicated the specificity of MPT51 aptamers towards its target, M.tb MPT51.Polyclonal anti-MPT51 antibody cross-reacted with CFP-10 and Ag85C whereas, monoclonal anti-MPT51 antibody exhibited very low binding to MPT51 as depicted in FIG. 7. These results pointed towards the superiority of these 4 MPT51 aptamers over their anti-MPT51 antibody. In-house anti-MPT51 polyclonal antibody was used while monoclonal was procured from BEI, USA.

### Example 5: Aptamer-Antibody Competition Assay

To determine the effect of anti-MPT51 poly and monoclonal antibody on binding of MPT51 aptamers, aptamer antibody competition assay was performed using ALISA format. For this assay, two different conditions were employed, aptamer favorable condition (in which all the dilutions and washing was done in selection buffer) and antibody favorable condition (in which all the dilutions and washing was done in PBS buffer). Briefly, anti-MPT51 antibody was diluted in the ratio of 1:5000 and 1:100 for polyclonal and monoclonal respectively in PBS (for antibody favorable condition) and selection buffer (for aptamer favorable condition) were added to M.tb MPT51-coated well (in a 96-well plate) along with 100 picomoles of biotinylated G-rich MPT51 aptamers namely SEQ ID No: 20 or (MPT51)20, SEQ ID No:23 or (MPT51)23, SEQ ID No:45 or (MPT51)45, and SEQ ID No:58 or (MPT51)58 in condition favorable respective buffers for 1 hour. Washing was given as per condition supplemented with 1% Tween-20. The plate was then incubated with Streptavidin-HRP that binds to 5′-biotin labeled MPT51 aptamer and developed with TMB as described in Step-4.

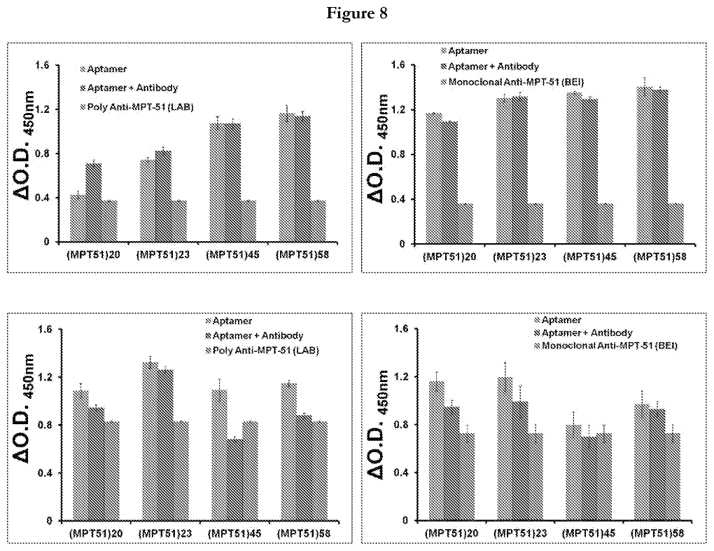

In FIG. 8 (A) where conditions favorable aptamer were used, binding of MPT51 aptamers remained unaltered in presence of poly or monoclonal antibody suggesting that either MPT51 aptamer has higher affinity for M.tb MPT51 in comparison to anti-MPT51 antibody or epitopes for MPT51 aptamer and anti-MPT51 antibody are different. However in case of antibody favoring condition where PBS is used as buffer, binding of MPT51 aptamers showed some reduction as depicted in FIG. 8B suggesting the requirement of potassium (‘K’) that is essential for the formation of Aptamer G Quadruplex which was absent in PBS.

These observations suggest that in near future these MPT51 aptamers can be used in combination with anti-MPT51 antibodies to develop a sandwich assay for diagnosis of tuberculosis.

### Example 6: Limit of Detection of MPT51 Using Aptamer (Determination of Limit of Detection)

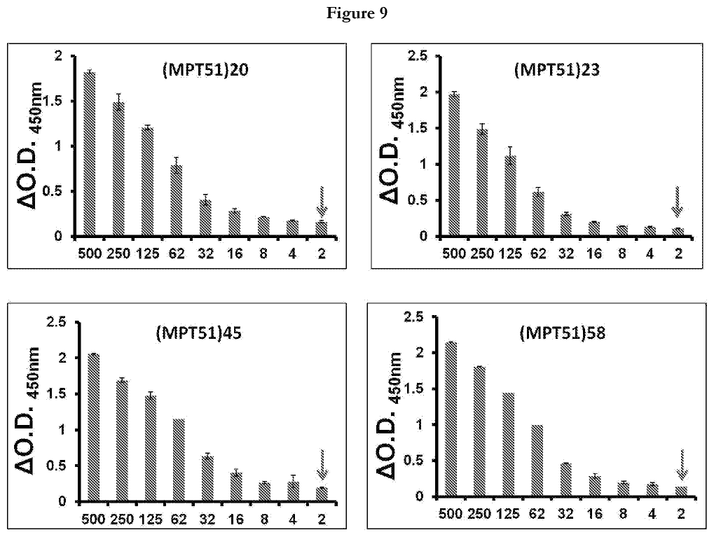

In order to determine the limit of detection (LOD) of MPT51 aptamers, the ALISA format was used. Different concentrations of M.tb MPT51 (2 ng-500 ng) was coated on a 96 well-ELISA plate. The ALISA procedure was performed subsequently as described in Example 2. O.D. was plotted as a function of M.tb MPT51 concentration at which the MPT51 aptamers displayed detection. ΔO.D. at 450 nm was plotted by taking mean+3SD of controls, which included primary control (no MPT51 aptamers), secondary control (no Streptavidin-HRP) and antigen control (no M.tb MPT51). The LOD of MPT51 aptamers was determined as the lowest amount of M.tb MPT51 giving absorbance after subtracting the O.D.450 value from mean+3SD of controls as depicted in FIG. 9.

The limit of detection was found to be 2 ng for SEQ ID No: 20 or (MPT51)20, SEQ ID No:23 or (MPT51)23, SEQ ID No:45 or (MPT51)45, and SEQ ID No:58 or (MPT51)58, which establishes high sensitivity of these aptamers towards M.tb MPT51.

### Example 7: Determination of Apparent Dissociation Constants (K_{d})

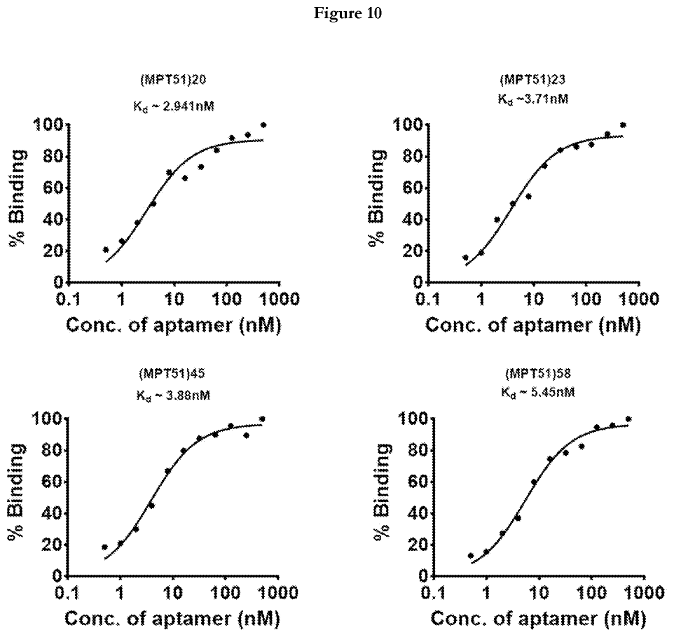

For determination of apparent dissociation constant (K_{d}) of SEQ ID No: 20 or (MPT51)20, SEQ ID No:23 or (MPT51)23, SEQ ID No:45 or (MPT51)45, and SEQ ID No:58 or (MPT51)58, 500 ng of M.tb MPT51/well was coated at 4° C. overnight on a 96 well plate in 100 μL of coating buffer. After blocking with 5% BSA supplemented with 0.25% Tween-20, a different concentration of MPT51 aptamers ranging from 0.5 nM to 500 nM were added. ALISA was performed as described in Step 7. O.D.450 was plotted as a function of MPT51 aptamer concentration and K_{d} was determined by non-linear regression for one-site binding using Graph-pad Prism version 5.02. The aptamers were ranked in terms of K_{d} (highest to lowest affinity) as (MPT51)20 or SEQ ID No:20 (2.9 nM) >SEQ ID No:23 or (MPT51)23 (3.71 nM) >SEQ ID No.45 or (MPT51)45 (3.88 nM) >SEQ ID No. 58 or (MPT51)58 (5.45 nM) as depicted in FIG. 10. Low nanomolar K_{d} indicates stable binding of MPT51 aptamers with M.tb MPT51.

### Example 8: Development of Electrochemical Sensor APTADx

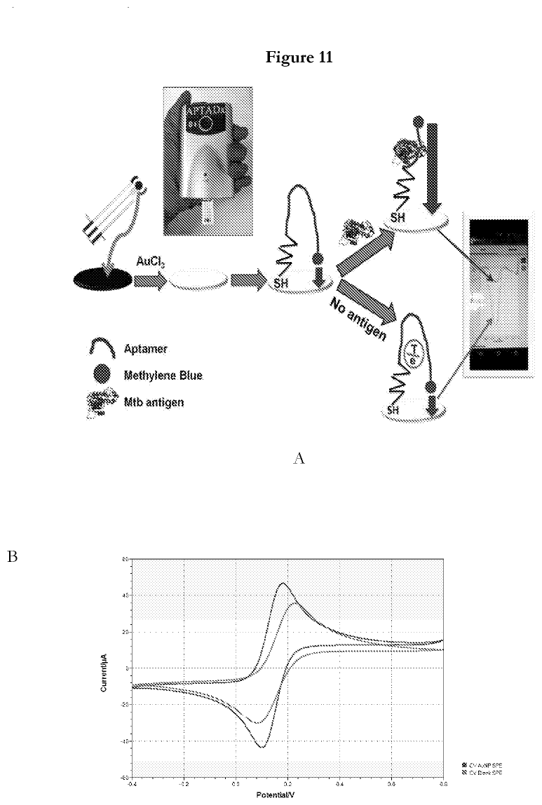

Inventors have designed an electrochemical sensor named APTADx using MPT51 aptamer (MPT51)45 or SEQ ID No: 45 for highly specific and ultrasensitive detection of M.tb MPT51. (MPT51)45 or SEQ ID No: 45 is used as probe for electrochemical sensing. Conformational changes in (MPT51)45 or SEQ ID No: 45 induced by binding of (MPT51)45 or SEQ ID No: 45 with M.tb MPT51 leads to the change in the distance between the labeled redox moieties and the electrode, which eventually results in the large variation in the voltammetric signal that could be used for electrochemical sensing of the targets. This aptasensor developed here follow the mechanism of “turn-off” sensors as depicted in FIG. 11A. In absence of M.tb MPT51 in biological sample, the small distance between working electrode and redox moiety is maintained that leads to very strong electron transfer, thus strong current flow (high signal). Whereas, in presence of M.tb MPT51 in biological sample, MPT51 aptamer undergo conformational changes on binding to M.tb MPT51 which increases distance between working electrode and redox moiety, thus only weak electron transfer is possible resulting significant decrease in current. This change in current can be monitored by a handheld Potentiostat and can easily be monitored and recorded in real time. This assay may also be controlled through an android based application that can disseminate the results of assay as soon as assay is done. APTADx demonstrated good sensitivity both in buffer and clinical specimen (CSF).

Fabrication of electrode: Electrochemical deposition of gold nanoparticle was carried out on carbon-based screen-printed electrode (SPE) using 1 mM HAuCl4 containing 0.5 M H2504 with potentiostatic method at −0.2 V for 120 seconds was and gently washed with distilled water and dried at room temperature. These SPE was further characterized before and after deposition of AuNPs. For electrochemical characterization, cyclic voltammogram (CV) was performed in 5 mM [Fe(CN)₆^{3-/4-}] in a potential range of −0.4V to 0.8V with scan rate 10 mV/S. This controlled electrochemical deposited AuNPs modified electrode surface serves as a platform for immobilization of thiol-modified aptamers, with increased sensitivity and loading capability of the sensor. The CVs of bare SPE and AuNPs modified electrodes represented in FIG. 11B, a pair of well-defined redox peaks was observed on the bare SPE (FIG. 11B curve a). After the electrodeposition of AuNPs, the peak current of [Fe(CN)₆^{3-/4-}] increased and the peak to peak potential separation (ΔEp) decreased slightly, indicating a better redox behavior of [Fe(CN)₆^{3-/4-}] on the AuNPs modified electrode (FIG. 11B curve b). The average value of the electroactive surface area of the unmodified and AuNPs modified electrodes were calculated according to the Randles-Sevick equation (Bard et al., 2000):

I p=2.69×105AD1/2n3/2γ1/2C

Where, n is the number of electrons participating in the redox reaction, A is the area of the electrode (cm2), D is the diffusion coefficient of the molecule in solution (cm2 s-1), C is the concentration of the probe molecule in the bulk solution (mol cm-3), and γ is the scan rate (V s-1). The electroactive surface of bare screen-printed electrode and AuNPs modified electrode are 10.22×10-2 cm2 and 13.33×10-2 cm2 respectively.

Immobilization of R45: Followed by electrochemical deposition of AuNPs, 5.0 μl of thiol activated by TCEP (tris(2-carboxyethyl) phosphine) (MPT51)45 or SEQ ID No: 45 (10 μM) in selection buffer (10 mM Tris-HCl pH 7.4 supplemented with 50 mM KCl, 10 mM MgCl₂ and 25 mM NaCl) was dropped on modified SPE. This electrode was incubated at 30° C. for overnight. Then the (MPT51)45 or SEQ ID No: 45 modified electrode was thoroughly rinsed with selection buffer followed by distilled water to remove the weakly adsorbed (MPT51)45 or SEQ ID No: 45 and dried. Thiolated (MPT51)45 or SEQ ID No: 45 modified electrode was further exposed with 10 mM 2-mercaptoethanol (β-ME) for 20 min to block the marginal sites on electrode surface. Finally, the modified electrode was rinsed thoroughly with selection buffer (10 mM Tris-HCl supplemented with 50 mM KCl, 25 mM NaCl and 10 mM MgCl₂) and double distilled water respectively.

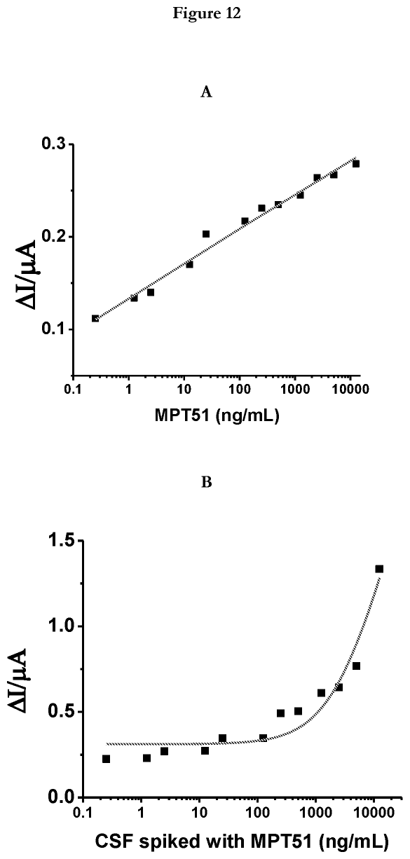

Detection of M.tb MPT51: Inventors of the present invention tested APTADx with sample containing 0.25 to 12500 ng/mL of M.tb MPT51.Sample containing M.tb MPT51 was incubated with (MPT51)45 or SEQ ID No: 45 modified SPE for 15 min at RT. After incubation, the (MPT51)45 or SEQ ID No: 45 modified electrodes were washed with 1× selection buffer carefully to remove the unbound M.tb MPT51. In order to detect bound M. tb MPT51, Differential pulse voltammetry (DPV) was performed in 1× selection buffer. FIGS. 12 & 13 represent the calibration curve of difference in reductive signal obtained before and after the incubation with recombinant M.tb MPT51. The signal measured is due to the methylene blue tagged to 5′ end of the (MPT51)45 or SEQ ID No: 45. The reduction peak current obtained from Methylene Blue is due to the electron transfer between the Methylene Blue tagged (MPT51)45 or SEQ ID No: 45 and electrode surface. In case of free (MPT51)45 or SEQ ID No: 45 i.e. before incubation with the M.tb MPT51 the reduction peak current is more when compared with after incubation with M.tb MPT51 and this signal reduces as the concentration of the M.tb MPT51 increases.

APTADx was challenged with a range of MPT51 concentration (0.25-12500 ng/mL). Using the developed sensor as low as 0.25 ng/mL antigen can be detected. FIG. 12 evince electrochemical detection of MPT51 using dual labeled (methylene blue and thiol labeled) aptamer using APTADx platform. Similarly, we have determined the low-end detection limit (LOD) for aptamer-based ECS in CSF background. To determine LOD in CSF background, a range of (0.25-12500 ng/mL) MPT51 antigen was spiked in CSF and sensor response was recorded in the aforementioned manner. FIG. 12A demonstrates the electrochemical detection of MPT51 using dual labeled (methylene blue and thiol labeled) aptamer using APTADx platform. In CSF background this sensor is able to detect as low as 0.25 ng/mL antigen (FIG. 12B)

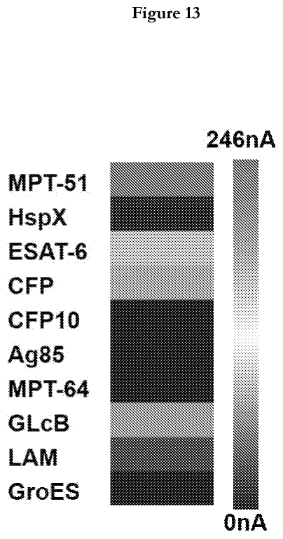

Selectivity of APTADx: In order to evaluate the selectivity of APTADx control experiments were performed using different M.tb antigenic proteins namely CFP-10, ESTAT-6, Ag85, GroES, MPT64, HspX, MPT51, CFP, and LAM. The (MPT51)45 or SEQ ID No: 45 modified electrode was incubated with 50 ng of these antigenic proteins for 15 minutes at RT. After incubation, the (MPT51)45 or SEQ ID No: 45 modified electrodes were washed with selection buffer carefully to remove the unbound antigenic protein and DPV was recorded in selection buffer. FIG. 13 represents the heatmap for the specificity of the sensor with above mentioned M.tb proteins. It was observed that there is no significant signal obtained on exposing with the M.tb proteins other than M.tb MPT51. These results demonstrated that APTADx has highly specific response for M.tb MPT51.

Testing Clinical Sample in APTADx: A subset of 55 pediatric CSF samples was derived from an archived set of samples from specimen bank of Prof. Jaya S. Tyagi's Lab (Co-inventor, AIIMS-Delhi), wherein the samples were categorized according to a universal case definition for TBM (Marais et al. 2012). The criteria defines ‘Definite’ TBM as culture/smear/commercial NAAT positive/AFB seen on autopsy and ‘Probable and Possible’ TBM groups include subjects negative by the above criteria but satisfying defined clinical criteria, CSF criteria, cerebral imaging criteria and evidence of extraneural TB having a score of ≥10-12 (‘Probable’ TBM) and a score of ≥6-11 (‘Possible’ TBM). In our CSF sample subset (n=55), samples were classified as ‘Definite’ TBM on the basis of M.tb culture positivity only (n=10), as ‘Probable’ TBM (n=6, score range: 10-18), ‘Possible’ TBM (n=10, score range: 6-9) and Not-tuberculous' meningitis with an alternative diagnosis established (n=29). The ‘Not-TBM’ category was further sub-divided into Non-tuberculous infectious meningitis (NTIM, n=10), Infectious neurological disorders (IND, n=10) and Non-infectious neurological disorders (NIND, n=9). The median age and range of the children in each diagnostic category were quite comparable. The NTIM comprised of cases of pyogenic bacterial meningitis that included culture confirmed cases of E. coli (n=1), and Acinetobacter sp (n=1). Other 8 cases were diagnosed on the basis of response to appropriate antibiotics, clinical presentation and symptoms. The IND category included 5 cases of meningoencephalitis, 4 cases of enteric encephalopathy, case of sepsis. The NIND category included 5 cases of neurodegenerative disorders, cases each of hypocalcemic seizures and transverse myelitis.

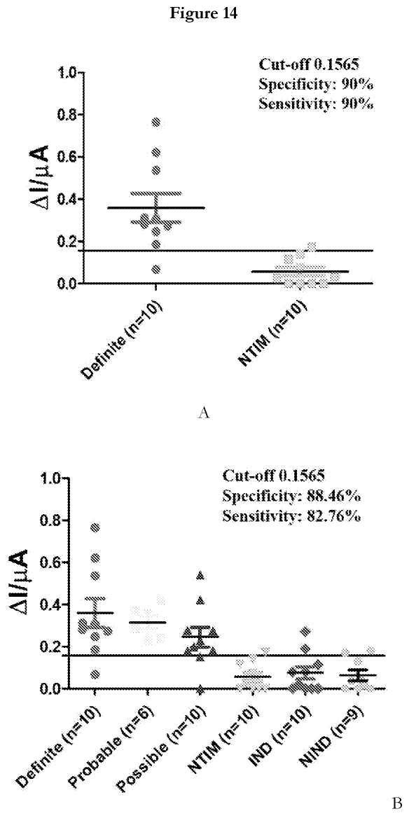

To detect M.tb MPT51 in CSF samples using APTADx, CSF samples were diluted at 1:10 dilution with selection buffer and incubated with (MPT51)45 or SEQ ID No: 45 modified SPE for 15 min at room temperature. Sensor response was recorded before and after addition of sample. FIGS. 14A and 14B representing that APTADx can efficiently discriminate between IBM and non-TBM specimens. A total 55 samples were tested that includes 29 non-TBM and 26 TBM samples. Based on the cut-off derived from the ROC curve using Definite (true positive n=10) and NTIM (true negative n=10) the performance of APTADx was evaluated (FIG. 13A). There was a highly significant (p<0.0001) difference between TBM and non-TBM group (FIG. 13B) was observed using developed sensor. This sensor yields a high sensitivity (˜82.76%) and specificity (′88.46%). This sample set is relatively smaller thus the sensitivity and specificity values may get better with large sample size.

We claim:

1. A single stranded DNA aptamer that binds to M.tb MPT51, wherein the single stranded DNA sequence or a complementary DNA sequence thereof, or a truncated portion thereof, or any pairing thereof, or any modification thereof, is homologous of any of: (a) SEQ ID NO: 1 to SEQ ID NO:80; or (b) a functional fragment of any preceding sequences.

2. A complex molecule comprising aptamer of claim 1 and a functional substance.

3. The complex molecule of claim 2, wherein the functional substance is selected from affinity substance and substance for labeling.

4. A diagnostic reagent comprising the aptamer of claim 1 or the complex of claims 2 and 3 for the detection of M.tb MPT51 in a biological sample.

5. A detection probe for M.tb MPT51 comprising the aptamer of claim 1 or the complex of claims 2 and 3.

6. An electrochemical device for the detection of M.tb MPT51 in a biological sample obtained from a subject suspected of M.tb infection using the aptamer of claim 1 or the diagnostic reagent of claim 4 or the detection probe of claim 5.

7. A method for detection of M.tb MPT51 in a biological sample obtained from a subject suspected of M.tb infection using the aptamer of claim 1 or the diagnostic reagent of claim 4 or the detection probe of claim 5 or the electrochemical device of claim 6, wherein tuberculosis in the subject is confirmed by the presence of M.tb MPT51 in the said biological sample.

8. The electrochemical device of claim 6 or the method of detection of claim 7, wherein the biological sample is selected from cerebrospinal fluid, sputum, pleural fluid, and ascitic fluid.
